(19) United States
(12) Patent Application Publication
(10) Pub. No.: US 2011/0117601 A1
(43) Pub. Date: May 19, 2011

(54) Glycosylation Profile Analysis

(76) Inventors: Markus Haberger, Munchen (DE); Dietmar Reusch, Muenchen (DE)

(21) Appl. No.: 12/674,980

(22) PCT Filed: Aug. 20, 2008
(86) PCT No.: PCT/EP08/06835

§ 371 Date: Feb. 24, 2010

(30) Foreign Application Priority Data
Aug. 31, 2007 (EP) 07017063.4

(51) Int. Cl. C12P 21/00

(52) U.S. Cl. 435/69.6; 435/69.1

(57) ABSTRACT

The present invention provides a method for the production of a glycosylated heterologous polypeptide comprising the steps of obtaining a sample from a crude fermentation broth, incubation of the sample with magnetic affinity beads, releasing glycans from the immobilized glycosylated polypeptides, measuring a glycosylation profile, comparing the glycosylation profile with a desired glycosylation profile of the recombinant glycosylated polypeptide, modifying the culture conditions in accordance to the glycosylation profile obtained, and repeating the process in order to obtain a glycosylated heterologous polypeptide with the desired glycosylation profile. With a similar method diagnostic markers can be identified and quantified.

The present invention relates to the field of recombinant proteins and their production. More particularly, the present invention relates to a method for the determination of the glycosylation profile of a recombinantly produced polypeptide, e.g. an antibody, and a process for the production of glycosylated polypeptides, wherein the glycosylation profile is determined during fermentation.

### BACKGROUND OF THE INVENTION

The glycosylation profile of a polypeptide is an important characteristic for many recombinantly produced therapeutic polypeptides. Glycosylated polypeptides, also termed glycoproteins, mediate many essential functions in eukaryotic organisms, e.g. humans, and some prokaryotes, including catalysis, signaling, cell-cell communication, activities of the immune system, as well as molecular recognition and association. They make up the majority of non-cytosolic proteins in eukaryotic organisms (Lis, H., et al., Eur. J. Biochem. 218 (1993) 1-27). The formation/attachment of oligosaccharides of a glycoprotein is a co- and posttranslational modification and, thus, is not genetically controlled. The biosynthesis of oligosaccharides is a multistep process involving several enzymes, which compete with each other for the substrate. Consequently, glycosylated polypeptides comprise a microheterogeneous array of oligosaccharides, giving rise to a set of different glycoforms containing the same amino acid backbone.

The covalently bound oligosaccharides do influence physical stability, folding, resistance to protease attack, interactions with the immune system, bioactivity, and pharmacokinetics of the respective polypeptide. Moreover some glycoforms can be antigenic, prompting regulatory agencies to require analysis of the oligosaccharide structures of recombinant glycosylated polypeptides (see e.g. Paulson, J. C., Trends Biochem. Sci. 14 (1989) 272-276; Jenkins, N., et al., Nature Biotech. 14 (1998) 975-981). Terminal sialylation of glycosylated polypeptides for example has been reported to increase serum-half life of therapeutics, and glycosylated polypeptides containing oligosaccharide structures with terminal galactose residues show increased clearance from circulation (Smith, P. L., et al., J. Biol. Chem. 268 (1993) 795-802). Thus, in the biotechnological production of therapeutic polypeptides such as immunoglobulins the assessment of oligosaccharide microheterogeniety and its batch-to-batch consistency are important tasks.

Monoclonal antibodies (mAbs) are one of the fastest growing classes of protein therapeutics. In 2005, a total of 31 mAb-based products had been accepted for human therapy, e.g. for treating cancer, autoimmune and inflammatory diseases, or in vivo diagnostics, and many more are now in clinical trials (Walsh, G., Trends Biotechnol. 23 (2005) 553-558). Antibodies differ significantly from other recombinant polypeptides in their glycosylation pattern. Immunoglobulin G (IgG) e. g. is a symmetrical, multifunctional glycosylated polypeptide of an approximate molecular mass of 150 kDa consisting of two identical Fab parts responsible for antigen binding and the Fc part for effector functions. Glycosylation tends to be highly conserved in IgG molecules at Asn-297, which is buried between the CH₂ domains of the Fc heavy chain, forming extensive contacts with the amino acid residues within CH₂ (Sutton and Phillips, Biochem. Soc. Trans. 11 (1983) 130-132). The Asn-297 linked oligosaccharide structures are heterogeneously processed, such that an IgG exist in multiple glycoforms. Variations exist in the site occupancy of the Asn-297 site (macroheterogeniety) or by variation in the oligosaccharide structure at the glycosylation site (microheterogeniety), see for example Jenkins, N., et al., Nature Biotechnol. 14 (1996) 975-981. Generally, the more abundant oligosaccharide groups in IgG mAb are asialo biantennary complex type glycans, primarily agalactosylated (G0), mono-galactosylated (G1), or bi-galactosylated (G2) types (Jefferis, R., et al., Immunol. Lett. 68 (1998) 47-52).

The oligosaccharides bound to the Fc region, do not only effect physicochemical properties (e.g. structural integrity) and abolish or minimize protease resistance but are also essential for effector functions, such as complement binding, binding to macrophage Fc receptors, rapid elimination of antigen-antibody complexes from the circulation, and induction of antibody-dependent cell-mediated cytotoxicity (ADCC) (Cox, K. M., et al., Nature Biotechnol. 24 (2006) 1591-1597; Wright and Morrison, Trends Biotechnol. 15 (1997) 26-32). Because different glycoforms can be associated with different biological properties, the ability to enrich for a specific glycoform may be useful, for example, to elucidate the relationship between a specific glycoform and a specific biological function. Thus, production of glycosylated polypeptide compositions that are enriched for particular glycoforms is highly desirable. Much research has been conducted to understand the effects of environmental factors and culture conditions on protein glycosylation and glycosylation pattern of proteins. Culture variables, like dissolved oxygen concentration (Kunkel, J. P., et al., J. Biotechnol. 62 (1998) 55-71), changes of monosaccharide availability (Tachibana, H., et al., Cytotechnology 16 (1994) 151-157), availability of intracellular nucleotide sugars (Hills, A. E., et al., Biotech. Bioeng. 75 (2001) 239-251), ammonium concentration (Gawlitzek, M., et al., Biotech. Bioeng. 68 (2000) 637-646), serum concentration (Parekh, R. B., et al., Biochem. J. 285 (1992) 839-845; Serrato, J. A., et al., Biotechnol. Appl. Biochem. 47 (2007) 113-124), and growth state (Robinson, D. K., et al., Biotech. Bioeng. 44 (1994) 727-735) have been reported to lead to differences in the glycosylation profile.

Chinese Hamster Ovary (CHO) cells are most commonly used for production of glycosylated polypeptides for therapeutical use. These cells produce a defined glycosylation profile and allow generation of genetically stable, highly productive cell lines. Moreover, they can be cultured to high cell densities in serum-free media for the development of safe and reproducible bioprocesses. The N-acetylglucosamine content and type of glycosylated polypeptides expressed in CHO cells has been affected by temperature and osmolality in the presence of alkanoic acid (see e. g. U.S. Pat. No. 5,705,364). In US patent application 2003/0190710 it has been reported that the mere adaptation to temperature and osmolality altered the level of the glycosylated heavy chain variant of an IgG in a CHO cell culture.

High performance anion exchange chromatography with pulsed amperometric detection (HPAEC) and matrix-assisted laser desorption ionization time-of-flight mass spectrometry (MALDI-TOF MS) have been used to analyze the carbohydrate moieties of glycosylated polypeptides (see e.g. Fukuda, M., (ed) Glycobiology: A Practical Approach, IRL Press, Oxford; Morelle, W., and Michalsky, J. C., Curr. Pharmaceut. Design 11 (2005) 2615-2645). Hoffstetter-Kuhn, S., et al. (Electrophoresis 17 (1996) 418-422) used capillary electrophoresis and MALDI-TOF MS analysis to profile the oligosaccharide-mediated heterogeneity of a monoclonal antibody after deglycosylation of the antibody with N-glycosidase F (PNGase F).

Given the importance of glycosylation on functional properties of recombinant glycosylated polypeptides and the necessity of a well-defined and consistent product production process, an on-line or ad-line analysis of the glycosylation profile of recombinantly produced glycosylated polypeptides during the fermentation process is highly desirable. Papac, D. I., et al., (Glycobiol. 8 (1998) 445-454) reported a method containing the immobilization of glycosylated polypeptides on a polyvinylidene difluoride membrane, the enzymatic digestion and MALDI-TOF MS analysis of the glycosylation profile. The analysis and the molecular characterization of recombinantly produced mAbs, including several chromatography steps, is reported in Bailey, M., et al., J. Chromat. 826 (2005) 177-187.

### SUMMARY OF THE INVENTION

It is an object of the present invention to provide a method for the on-line analysis of the glycosylation profile of a recombinantly produced glycosylated polypeptides during fermentation in order to obtain said recombinantly produced polypeptide with a desired glycosylation profile.

One aspect of the current invention is a method for the recombinant production of a glycosylated heterologous polypeptide comprising the steps of:
(A) providing a cell comprising a nucleic acid encoding said heterologous polypeptide,
(B) cultivating the cell of (A) at defined culture conditions, suitable for the expression of the heterologous polypeptide,
(C) obtaining a sample from the cultivation medium,
(D) contacting the sample with magnetic affinity beads under conditions suitable for the binding of the heterologous polypeptide to the beads,
(E) releasing the glycans from the heterologous polypeptide bound to the magnetic affinity beads without releasing the heterologous polypeptide,
(F) purifying the released glycans of (E),
(G) determining the glycosylation profile of the heterologous polypeptide by analyzing the released and purified glycans of (F),
(H) comparing the determined glycosylation profile with a reference glycosylation profile,
(I) adjusting the culture conditions in accordance with the result obtained in step (H), optionally continuing with culturing, and
(J) repeating steps (C) to (H) to obtain the glycosylated heterologous polypeptide,
(K) recovering the glycosylated heterologous polypeptide from the culture medium or the cells.

In one embodiment the glycosylated heterologous polypeptide is an immunoglobulin, preferably a monoclonal immunoglobulin. In another embodiment is protein A, G, or L bound to the magnetic affinity beads as affinity ligand for selectively binding immunoglobulins employed in step (D). According to further embodiments, the glycans in step (E) are released enzymatically or chemically, e.g. by hydrazinolysis. In one embodiment, the glycans are released by treatment with an N-glycosidase. In a further embodiment, the glycans are purified in step (F) by reverse phase chromatography or cation exchange chromatography or a combination thereof. In a further embodiment, the glycosylation profile of the purified glycans obtained in step (E) is in step (G) determined by MALDI-TOF MS analysis or quantitative HPLC separation. In a further embodiment, steps (D) to (G) are performed in a high-throughput format using microtiter plates. In another embodiment the adjusted culture conditions in step (I) comprise alterations in (i) the concentration of one or more of nutrients, carbohydrates, additives, buffer compounds, ammonium, or dissolved oxygen, or (ii) the osmolality, the pH value, the temperature, or the cell density, or (iii) the growth state. In one embodiment is said heterologous polypeptide after step (K) subjected to a step (L) purifying said heterologous polypeptide. In another embodiment is said heterologous polypeptide secreted into the culture medium.

A second aspect of the present invention is to provide a method suitable for determining and/or quantifying a glycosylation marker comprising the steps of:
(A) contacting a sample containing a glycosylated polypeptide with magnetic affinity beads,
(B) releasing the glycans from the affinity bound glycosylated polypeptide without the release of the glycosylated polypeptide,
(C) purifying the released glycans,
(D) determining the glycosylation marker amount, and
(E) comparing the glycosylation marker amount with a reference amount.

In one embodiment is said sample a sample of a subject, preferably a mammal, more preferably of a human, most preferably of a patient. In another embodiment comprises the method prior to step (A) the step (A-1) treating a sample obtained from a subject by applying the sample to one or more chromatography columns and recovering the glycosylated heterologous polypeptide.

### DETAILED DESCRIPTION OF THE INVENTION

The present invention provides a method for the recombinant production of a glycosylated heterologous immunoglobulin with a desired glycosylation profile comprising the steps of:
(A) providing a mammalian cell, which has been transfected with a nucleic acid comprising a further nucleic acid encoding said heterologous immunoglobulin,
(B) cultivating the mammalian cell of step (A) under culture conditions, suitable for the expression of the heterologous immunoglobulin encoded by the further nucleic acid in a glycosylated form,
(C) obtaining a sample from the cultivation containing the glycosylated heterologous immunoglobulin,
(D) contacting the sample of step (C) with magnetic affinity beads, to which protein A, G, or L is chemically bound, under conditions suitable for the binding of the glycosylated heterologous immunoglobulin to the beads,
(E) obtaining the glycans from the bound heterologous immunoglobulin without the release of the heterologous immunoglobulin from the magnetic affinity beads,
(F) purifying the glycans obtained in step (E),
(G) determining the glycosylation profile of the heterologous immunoglobulin by determining the structure and composition of the purified glycans of step (F),
(H) comparing the determined glycosylation profile of step (G) with a reference glycosylation profile,
(I) adjusting the culture conditions in accordance with the result obtained in step (H), and
(J) if the culturing is continued repeating steps (C) to (H), or
(K) recovering the glycosylated heterologous polypeptide with the desired glycosylation profile from the cells or the cultivation medium.

It was surprisingly found that the method according to the invention enables the on-line follow up and adjustment of the bioprocess unit operations in order to influence the glycosylation profile of the produced heterologous polypeptide during the same cultivation process from which the sample has been obtained. This is important e. g. with regard to product consistency, therapeutic efficacy, and/or tolerability of e. g. a recombinantly produced immunoglobulin. In one embodiment comprises step (E) the cleavage of the glycans from the heterologous polypeptide and the recovery of the cleaved glycans without the release of the heterologous polypeptide from the magnetic affinity beads by removing the magnetic affinity beads with the bound heterologous immunoglobulin.

The practice of the present invention will employ conventional techniques of molecular biology, microbiology, recombinant DNA techniques, and immunology, which are within the skills of an artisan in the field. Such techniques are reported in the literature. See e.g., Sambrook, Fritsch & Maniatis, Molecular Cloning; a Laboratory Manual (1989); DNA Cloning, Volumes I and II (D. N. Glover, ed., 1985); Oligonucleotide Synthesis (Gait, M. J., ed., 1984); Nucleic acid Hybridization (Hames, B. D. & Higgins, S. J. eds., 1984); Transcription and translation (Harnes, B. D. & Higgins, S. J., eds., 1984); Animal cell culture (Freshney, R. L., ed., 1986); Immobilized cells and enzymes (IRL Press, 1986); Perbal, B., A practical guide to molecular cloning (1984); the series, Methods in Enzymology (Academic Press, Inc.); Gene transfer vectors for mammalian cells (J. H. Miller and M. P. Calos eds., 1987, Cold Spring Harbot Laboratory), (Wu, R., and Grossman, L., Methods in Enzymology 154 (1987) and Wu, R, Methods in Enzymology 155 (1987); Immunochemical methods in cell and molecular biology (Mayer and Walker, eds., 1987, Academic Press, London), Scopes, Protein purification: Principles and practice, second Edition (1987, Springer-Verlag, N.Y.); and Handbook of experimental immunology, Volumes I-IV (D. M. Weir and C. C. Blackwell eds., 1986).

The following terms, unless otherwise indicated, shall be understood to have the following meanings:

The term “polysaccharide” denotes molecules which are composed of a chain of monosaccharide units linked by glycosidic bonds. The distinction between “polysaccharides” and “oligosaccharides” is based upon the number of monosaccharide units present in the chain. Oligosaccharides typically contain between two and nine monosaccharide units, and polysaccharides contain ten or more monosaccharide units. In the current invention the term “polysaccharide” encompasses molecules consisting of two or more monosaccharide units, especially are encompassed molecules wherein the longest chain of monosaccharides is between three and nine monosaccharide units. The term “polysaccharides” encompasses linear and branched molecules, isolated as well as polypeptide bound molecules, sialylated and non-sialylated molecules.

The term “monosaccharide” denotes a simple sugar. Such a simple sugar may comprise from three to ten carbon atoms, preferably of from five to seven carbon atoms, it may be an aldose or ketose, and it may be in D- or L-configuration compared to D- or L-glyceraldehyde. Monosaccharides are for example threose, erythrose, or erythrulose (four carbon atoms), or arabinose, xylose, ribose, lyxose, ribulose, or xylulose (five carbon atoms), or allose, glucose, fructose, maltose, mannose, galactose, fucose, gulose, idose, altrose, talose, psicose, sorbose, or tagatose (six carbon atoms), mannoheptulose or sedoheptulose (seven carbon atoms), or sialose (nine carbon atoms). Preferably the term monosaccharide denotes ribose, glucose, fructose, fucose, maltose, galactose, and mannose.

The term “glycan” refers to a polymer that consists of monosaccharide residues. Glycans can be linear or branched. Glycans can be found covalently linked to non-saccharide moieties, such as lipids or proteins. Binding to proteins occurs via N- or O-linkages. The covalent conjugates comprising glycans are termed e. g. glycosylated polypeptides, glycoproteins, glycopeptides, peptidoglycans, proteoglycans, glycolipids, and lipopolysaccharides. Besides the glycans being found as part of a glycoconjugate, glycans exist also in free form (i.e., separate from and not associated with another moiety).

The terms “glycosylated polypeptide” and “glycoprotein” which are used interchangeably within this application refer to polypeptides or proteins having more than ten amino acids wherein at least one amino acid has a covalently attached polysaccharide. Preferably the polysaccharide is either bound via the OH group of a serine or a threonine (O-glycosylated polypeptide) or via the amide group (NH₂) of asparagine (N-glycosylated polypeptide). The glycoproteins may be homologous to the host cell, or preferably, heterologous, i.e., foreign, to the host cell expressing it, such as e.g. a human protein produced by a CHO cell.

The term “glycosylation” means the attachment of polysaccharides to a polypeptide. Preferably the polysaccharide consists of from two to twelve simple sugars linked together via glycosidic bonds.

The term “N-linked glycosylation” refers to the attachment of the polysaccharide to an asparagine residue of an amino acid chain. The skilled artisan will recognize that, for example, murine IgG1, IgG2a, IgG2b and IgG3 as well as human IgG1, IgG2, IgG3, IgG4, IgA and IgD C_{H}2 domains each have a single site for N-linked glycosylation at amino acid residue 297 (numbering according to Kabat, E. A., et al., Sequences of Proteins of Immunological Interest, 1991).

The term “O-linked glycosylation” refers to the attachment of the carbohydrate moiety to a serine or threonine residue of an amino acid chain.

The terms “glycoprofile” or “glycosylation profile” which are used interchangeably within this application refer to the properties of the glycans of a glycosylated polypeptide. These properties are preferably the glycosylation sites, or the glycosylation site occupancy, or the identity, structure, composition or quantity of the glycan and/or non-saccharide moiety of the polypeptide, or the identity and quantity of the specific glycoform.

The term “under conditions suitable for binding” and grammatical equivalents thereof as used within this application denotes that a substance of interest, e.g. PEGylated erythropoietin or an antibody, binds to a stationary phase when brought in contact with it, e.g. an ion exchange material. This does not necessarily denote that 100% of the substance of interest is bound, but essentially 100% of the substance of interest is bound, i.e. at least 50% of the substance of interest is bound, preferably at least 75% of the substance of interest is bound, preferably at least 85% of the substance of interest is bound, more preferably more than 95% of the substance of interest is bound to the stationary phase.

The term “glycoform” denotes a type of polypeptide with a specific type and distribution of polysaccharides attached to, i.e. two polypeptides would be of the same glycoform if they comprise glycans with the same number, kind, and sequence of monosaccharides, i.e. have the same “glycosylation profile”.

The term “host cell” covers any kind of cellular system which can be engineered to generate modified glycoforms of proteins, protein fragments, or peptides of interest, including immunoglobulins and immunoglobulin fragments. Preferably the host cell is a eukaryotic cell. More preferably the eukaryotic cell is a mammalian cell. Most preferably the host cell is a CHO, BHK, PER.C6® cell or HEK293 cell.

The terms “antibody”, “immunoglobulin”, “IgG” and “IgG molecule” are used interchangeably within this application. The term “immunoglobulin” encompasses the various forms of antibody structures including but not being limited to whole antibodies, antibody fragments, or antibody conjugates, and refers to a protein comprising one or more polypeptides substantially or partially encoded by immunoglobulin genes or fragments of immunoglobulin genes. The term antibody is used to denote whole antibodies and antigen binding fragments thereof. The recognized immunoglobulin genes include the kappa (κ), lambda (λ), alpha (α), gamma (γ), delta (δ), epsilon (ε), and mu (μ) constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. A typical immunoglobulin (e.g. antibody) structural unit is a tetramer. Each tetramer is composed of two pairs of polypeptide chains, each pair having one “light” (about 25 KDa) and one “heavy” chain (about 50-70 KDa). The N-terminus of each chain defines a variable region of about 100 to 120 or more amino acids primarily responsible for antigen binding. The terms variable light chain (VL) and variable heavy chain (VH) refer to the light and heavy chain variable domains, respectively.

Immunoglobulins also include single-armed composite monoclonal antibodies, single chain antibodies, including single chain Fv (scFv) antibodies in which a variable heavy and a variable light chain are joined together (directly or through a peptide linker) to form a continuous polypeptide, as well as diabodies, tribodies, and tetrabodies (Pack, P., et al., J. Mol. Biol. 246 (1995) 28-34; Pack, P., et al., Biotechnol. 11 (1993) 1271-1277; Pack, P., et al., Biochemistry 31 (1992) 1579-1584). The antibodies are, e.g., polyclonal, monoclonal, chimeric, humanized, single chain, Fab fragments, fragments produced by a Fab expression library, or the like. Preferably the antibody or antibody fragment or antibody variant is a monoclonal antibody.

The terms “monoclonal antibody” (mAb) or “monoclonal antibody composition” as used herein refer to a preparation of antibody molecules produced by a single cell and/or its progeny by cultivation.

The terms “cell,” “cell line,” and “cell culture” are used interchangeably and all such designations include progeny. Thus, the words “transformants” and “transformed cells” include the primary subject cell and cultures derived there from without regard for the number of transfers. It is also understood that all progeny may not be precisely identical in DNA content, due to deliberate or inadvertent mutations. Variant progeny that have the same function or biological activity as screened for in the originally transformed cell are included.

The terms “expression” or “expresses” refer to transcription and translation occurring within a host cell. The level of expression of a product gene in a host cell may be determined on the basis of either the amount of corresponding mRNA that is present in the cell or the amount of the polypeptide encoded by the structural gene that is expressed in the cell.

The term “cultivation” or “cultivation medium” as use within this application denotes the entire content of the vessel wherein the fermentation of the host cell, i.e. the production of the heterologous polypeptide, is carried out. This comprises in addition to the produced heterologous polypeptide, other proteins and protein fragments present in the medium, e.g. from the added nutrients or from dead cells, host cells, cell fragments, and all constituents supplied with the nutrient medium and produced by the host during the cultivation.

The term “recombinant” when used with reference, e.g., to a cell, polynucleotide, vector, protein, or polypeptide typically indicates that the cell, polynucleotide, or vector has been modified by the introduction of a heterologous (or foreign) nucleic acid or the alteration of a native nucleic acid, or that the protein or polypeptide has been modified by the introduction of a heterologous amino acid, or that the cell is derived from a cell modified by the introduction of heterologous nucleic acid. Recombinant cells express heterologous polypeptides or nucleic acids that are not found in the native (non-recombinant) form of the cell or express native nucleic acid sequences that would otherwise be abnormally expressed, under-expressed, or not expressed at all. The term “recombinant” when used with reference to a cell indicates that the cell comprises a heterologous nucleic acid and/or expresses a polypeptide encoded by a heterologous nucleic acid. Recombinant cells can contain coding sequences that are not found within the native (non-recombinant) form of the cell. Recombinant cells can also contain coding sequences found in the native form of the cell wherein the coding sequences are modified and/or re-introduced into the cell by artificial means. The term also encompasses cells that contain a nucleic acid endogenous to the cell that has been modified without removing the nucleic acid from the cell; such modifications include those obtained by gene replacement, site-specific mutation, recombination, and related techniques.

The term “additive” refers to constituents of the culture medium which are not essential for the cells to grow but is, for example, added to the culture medium in order to enhance growth or survival of the cells or to change the glycosylation profile of a glycosylated polypeptide produced by recombinant cells cultivated in said culture medium.

The term “nutrient” refers to constituents of the cultivation medium which are essential for the cells to grow and/or to survive.

The term “subject” means an animal, more preferably a mammal, and most preferably a human.

The carbohydrate moieties of the present invention will be described with reference to commonly used nomenclature for the description of oligosaccharides. A review of carbohydrate chemistry which uses this nomenclature is found in Hubbard, S. C., and Ivatt, R. J., Ann. Rev. Biochem. 50 (1981) 555-583.

One aspect of the current invention is a method for the recombinant production of a glycosylated heterologous polypeptide in a cultivation medium comprising the steps of:
(A) providing a cell comprising at least one nucleic acid encoding said glycosylated heterologous polypeptide, in one embodiment comprising two nucleic acids encoding said glycosylated heterologous polypeptide,
(B) incubating said cell under predetermined cultivation conditions in a serum-free cultivation medium, whereby said glycosylated heterologous polypeptide is obtained in glycosylated form in said cultivation medium,
(C) obtaining a sample from the cultivation medium, preferably not comprising cells,
(D) contacting said sample with magnetic affinity beads, thereby binding the glycosylated heterologous polypeptide to said magnetic affinity beads,
(E) releasing the glycans from the glycosylated heterologous polypeptide bound to the magnetic affinity beads, without the polypeptide being released from the magnetic affinity beads,
(F) purifying the glycans released in (E) by a liquid chromatography, in one embodiment by a high performance liquid chromatography on a cation exchange resin and/or on a reversed phase,
(G) determining the glycosylation profile of the glycosylated polypeptide by analyzing the purified glycans obtained in (F) by Matrix Assisted Laser Desorption/Ionisation Time Of Flight mass spectrometry (MALDI-TOF MS),
(H) comparing the glycosylation profile with a pre-determined reference glycosylation profile,
(I) if the glycosylation profile determined in (G) differs from the pre-determined reference glycosylation profile modifying the cultivation conditions in accordance with the results obtained in step (H) and repeating steps (C) to (H) to obtain the glycosylated heterologous polypeptide with a glycosylation profile according to the reference glycosylation profile or terminating the culturing.
(K) recovering the glycosylated heterologous polypeptide.

In one embodiment of the method of the current invention in step (A) the cell is a recombinant cell capable of expressing the heterologous polypeptide.

The heterologous polypeptide of interest can be produced either (a) by expression of a natural endogenous gene, or (b) by expression of an activated endogenous gene, or (c) by expression of an exogenous gene. In one embodiment of the invention the glycosylated heterologous polypeptide is recombinantly produced. Recombinant production methods and techniques are familiar to a person skilled in the art. This method e.g. comprises the production/providing of a nucleic acid(s) encoding the heterologous polypeptide, the introduction of said nucleic acid(s) in one (or more) expression construct(s), and the transfection of a host cell with said expression construct(s). Such an expression construct (vector) contains all regulatory elements required in addition to the coding nucleic acid(s) which are necessary for the expression of the heterologous polypeptide in the host cell. The host cell is cultured “under conditions suitable for the expression of” the heterologous polypeptide and the glycosylated heterologous polypeptide is isolated from the cells or the culture supernatant/cultivation medium.

The method according to the current invention is suitable for the production of any glycosylated heterologous polypeptide in a eukaryotic host cell. The method according to the invention is particularly suitable for the production of polypeptides that can be used therapeutically. For example the heterologous polypeptide can be selected from the group of polypeptides comprising immunoglobulins, immunoglobulin fragments, immunoglobulin conjugates, antifusogenic peptides, lymphokines, cytokines, hormones (e.g. EPO, thrombopoietin (TPO)), G-CSF, GM-CSF, interleukins, interferons, blood coagulation factors and tissue plasminogen activators. In one embodiment the heterologous polypeptide is selected from the group of polypeptides comprising immunoglobulins, immunoglobulin fragments, and immunoglobulin conjugates.

Cells useful in the method according to the invention for the production of a glycosylated heterologous polypeptide can in principle be any eukaryotic cells such as e.g. yeast cells or insect cells, as long as that cell attaches glycans to the heterologous polypeptide in order to obtain a glycosylated heterologous polypeptide. However, in one embodiment of the invention the eukaryotic cell is a mammalian cell. Preferably said mammalian cell is a CHO cell line, or a BHK cell line, or a HEK293 cell line, or a human cell line, such as PER.C6®. Furthermore, in one embodiment of the invention the eukaryotic cells are continuous cell lines of animal or human origin, such as e.g. the human cell lines HeLaS3 (Puck, T. T., et al., J. Exp. Meth. 103 (1956) 273-284), Namalwa (Nadkarni, J. S., et al., Cancer 23 (1969) 64-79), HT1080 (Rasheed, S., et al., Cancer 33 (1973) 1027-1033), or cell lines derived there from.

In one embodiment the immunoglobulins produced with the method according to the invention are recombinant immunoglobulins. In other embodiments the immunoglobulins are humanized immunoglobulins or chimeric immunoglobulins. Recombinant production of immunoglobulins is well-known in the art and described, for example, in the articles of Makrides, S. C., Protein Expr. Purif. 17 (1999) 183-202; Geisse, S., Protein Expr. Purif. 8 (1996) 271-282; Kaufman, R. J., Mol. Biotechnol. 16 (2000) 151-161; and Werner, R. G., Drug Res. 48 (1998) 870-880. For immunoglobulin production one or more nucleic acids encoding the light and heavy chains or fragments thereof are inserted into expression vectors by standard methods. Expression is performed in appropriate eukaryotic host cells like in the state of the art, such as e.g. CHO cells, NS0 cells, SP2/0 cells, HEK293 cells, COS cells, or yeast cells. The antibody is in one embodiment recovered from the cell or the cell supernatant after lysis or the cultivation medium.

Expression in NS0 cells is described by, e.g., Barnes, L. M., et al., Cytotechnology 32 (2000) 109-123; and Barnes, L. M., et al., Biotech. Bioeng. 73 (2001) 261-270. Transient expression is described by, e.g., Durocher, Y., et al., Nucl. Acids. Res. 30 (2002) E9. Cloning of variable domains is described by Orlandi, R., et al., Proc. Natl. Acad. Sci. USA 86 (1989) 3833-3837; Carter, P., et al., Proc. Natl. Acad. Sci. USA 89 (1992) 4285-4289; and Norderhaug, L., et al., J. Immunol. Meth. 204 (1997) 77-87. A transient expression system (HEK 293) is described by Schlaeger, E. J., and Christensen, K., Cytotechnology 30 (1999) 71-83; and by Schlaeger, E. J.; Immunol. Methods 194 (1996) 191-199.

In step (B) of the method according to the invention the cell is cultivated at defined or predetermined culture conditions, whereby, the glycosylated heterologous polypeptide is expressed. The term “predetermined culture conditions” as used within the current application denotes cultivation conditions which have been developed for the cultivation of a host cell for producing a glycosylated heterologous polypeptide with a defined glycosylation profile. The recombinant cell clones can be cultured generally in any desired manner. The nutrients added according to this aspect of the invention comprise essential amino acids, such as e.g. glutamine, or tryptophan, or/and carbohydrates, and optionally non-essential amino acids, vitamins, trace elements, salts, or/and growth factors such as e.g. insulin. In certain embodiments, the nutrients include at least one essential amino acid and at least one carbohydrate. These nutrients are metered in certain aspects of the invention into the fermentation culture in a dissolved state. In one embodiment, the nutrients are added over the entire growth phase (cultivation) of the cells, i.e. depending on the concentration of the selected parameters measured in the culture medium (this is termed fed-cultivation).

The cell culture according to the present invention is prepared in a medium suitable for the cultured cell. In one embodiment of the invention, the cultured cell is a CHO cell. Suitable culture conditions for mammalian cells are known (see e.g. Cleveland, W. L, et al., J. Immunol. Methods 56 (1983) 221-234). Moreover, the necessary nutrients and growth factors for the medium, including their concentrations, for a particular cell line, can be determined empirically without undue experimentation as described, for example, in “Mammalian cell culture”, Mather (ed., Plenum Press: NY, 1984); Animal cell culture: A Practical Approach, 2nd Ed; Rickwood, D. and Hames, B. D., eds., Oxford University Press: New York, 1992; Barnes, D., and Sato, G., Cell, 22 (1980) 649.

The term “under conditions suitable for the expression” denotes conditions which are used for the cultivation of a cell expressing a glycosylated heterologous polypeptide and which are known to or can easily be determined by a person skilled in the art. It is known to a person skilled in the art that these conditions may vary depending on the type of cell cultivated and type of polypeptide expressed. In general the cell is cultivated at a temperature, e.g. between 20° C. and 40° C., and for a period of time sufficient to allow effective production of the conjugate, e.g. for of from 4 to 28 days, in a volume of 0.01 to 10⁷ liter.

A “polypeptide” is a polymer consisting of amino acids joined by peptide bonds, whether produced naturally or synthetically. Polypeptides of less than about 20 amino acid residues may be referred to as “peptides”, whereas molecules consisting of two or more polypeptides or comprising one polypeptide of more than 100 amino acid residues may be referred to as “proteins”. A polypeptide may also comprise non-amino acid components, such as carbohydrate groups/glycans, metal ions, or carboxylic acid esters. The non-amino acid components may be added by the cell, in which the polypeptide is expressed, and may vary with the type of cell. Polypeptides are defined herein in terms of their amino acid backbone structure or the nucleic acid encoding the same. Additions such as carbohydrate groups are generally not specified, but may be present nonetheless.

The nutrient solution may in one embodiment be supplemented with one or more components from the following categories: plasma components, growth factors such as, e.g., insulin, transferrin, or EGF, hormones, salts, inorganic ions, buffers, nucleosides and bases, protein hydrolyzates, antibiotics, lipids, such as, e.g., linoleic acid. In one embodiment said nutrient solution is animal serum-free.

In one embodiment of the invention, the culture is a suspension culture. Furthermore, in another embodiment the cells are cultured in a medium containing low serum content, such as, e.g., a maximum of 1% (v/v). In a preferred embodiment the culture is a serum-free culture, e.g. in a serum-free, low-protein fermentation medium (see e.g. WO 96/35718). Commercially available media such as Ham's F10 or F12 (Sigma), Minimal Essential Medium (MEM, Sigma), RPMI-1640 (Sigma), or Dulbecco's Modified Eagle's Medium (DMEM, Sigma), containing appropriate additives are exemplary nutrient solutions. Any of these media may be supplemented as necessary with components as mentioned above.

The process according to the invention permits a culture in a culture volume of more than 1 l, preferably more than 10 l, preferably 50 l to 10,000 l. Furthermore the process according to the invention allows a high cell density fermentation, which denotes that the concentration of the cells after the growth phase (i.e. at the time of harvest) is more than 1×10⁶ cells/ml, in one embodiment more than 5×10⁶ cells/ml, or with a dry cell weight of more than 100 g/l, in one embodiment more than 200 g/l.

Cell culture procedures for the large- or small-scale production of glycosylated polypeptides are potentially useful within the context of the present invention. Procedures including, but not limited to, a fluidized bed bioreactor, hollow fiber bioreactor, roller bottle culture, or stirred tank bioreactor system may be used, in the latter two systems, with or without microcarriers. The systems can be operated in one of a batch, a fed-batch, a split-batch, a continuous, or a continuous-perfusion mode. In certain embodiments of the invention, the culture is carried out as a split-batch process with feeding according to requirements of the culture in which a portion of the culture broth is harvested after a growth phase and the remainder of the culture broth remains in the fermenter which is subsequently supplied with fresh medium up to the working volume. The process according to the invention enables the desired glycosylated polypeptide to be harvested in very high yields. Hence the concentration at the time of harvest is for example at least 300 mg/l, in one embodiment 500 mg/l, in one embodiment 1000 mg/l, and in another embodiment 1500 mg/l.

According to another aspect of the invention, fed-batch or continuous cell culture conditions are devised to enhance growth of the mammalian cells in the growth phase of the cell culture. In the growth phase cells are grown under conditions and for a period of time that is maximized for growth. Culture conditions, such as temperature, pH, dissolved oxygen (DO₂), etc., are those used with the particular host and are known to the skilled person. Generally, the pH is adjusted to a level between about 6.5 and 7.5 using either an acid (e.g. CO₂) or a base (e.g. Na₂CO₃ or NaOH) or a HEPES (N-2-hyxdroxyehylpiperazin-N′-2-ethane-sulfonic acid) based buffered system, buffered further with NaHCO₃ and adjusted with diluted NaOH. A suitable temperature range for culturing mammalian cells such as CHO cells is between about 20 to 40° C., in one embodiment between 25 and 38° C., in another embodiment between 30 and 37° C. In one embodiment the pO₂ is between 5-90% of air saturation. The osmolality can be regulated by changes in the concentrations of sodium chloride, amino acids, hydrolyzates, or sodium hydroxide and has a value of 320 to 380 mOsm in one aspect of the invention.

According to the present invention, the cell-culture environment during the production phase of the cell culture is controlled. The culture conditions for the glycosylated polypeptides to be produced are defined by the following parameter:
1. Basic medium:
   concentrations and types of nutrients, optional plasma components, growth factors, salts and buffers, nucleosides and bases, protein hydrolyzates, antibiotics and lipids, suitable carriers,
2. Parameter known to alter the glycosylation profile:
   types and concentrations of carbohydrate, dissolved oxygen, ammonium concentration, pH value, osmolality, temperature, cell density, growth state
3. Optionally further additives.

Further additives are for example non-essential compounds stimulating either cell growth and/or enhancing cell survival and/or manipulating the glycosylation profile of the glycosylated polypeptide in any desired direction. Additives comprise serum components, growth hormones, peptide hydrolyzates, small molecules (like dexamethason, cortisol, iron chelating agents, etc.), inorganic compounds (like Selene etc.), and compounds known to have an effect of the glycosylation profile (like butyrate or quinidine (see e.g. U.S. Pat. No. 6,506,598), alkanoic acid (U.S. Pat. No. 5,705,364), or copper (EP 1 092 037)). In one aspect all of the above under items 1, 2 and 3 listed parameters and compounds are serum free parameters, in another embodiment animal component derived free parameters.

In one aspect of the invention, the carbohydrates are monosaccharides and/or disaccharides such as glucose, glucosamine, ribose, fructose, galactose, mannose, sucrose, lactose, mannose-1-phosphate, mannose-1-sulfate, or mannose-6-sulfate. In one aspect of the invention, the total concentration of all sugars during the fermentation is of from 0.1 g/l to 10 g/l, in one embodiment of from 2 g/l to 6 g/l in the culture medium. The carbohydrate mixture is added dependent on the respective requirement of the cells (see e.g. U.S. Pat. No. 6,673,575).

The ammonium concentration is altered by adding NH₄Cl to the culture medium (Gawlitzek, M., et al., Biotech. Bioeng. 68 (2000) 637-646).

In step (C) of the method according to the current invention for the production of a recombinant glycosylated polypeptide, a sample is obtained from the crude fermentation broth and in step (D) of the method, the sample is incubated with magnetic affinity beads.

The glycosylated polypeptide of interest is recovered from the culture medium using techniques which are well established in the art. In certain embodiments of the invention, the glycosylated polypeptide of interest is recovered from the culture medium as a secreted polypeptide, or from host cell lysates.

If the glycosylated polypeptide of interest is a heterologous polypeptide the magnetic affinity beads can be selected that only the heterologous polypeptides binds and is thus separated from the other polypeptides from the cultivation in a single step. Thus, in one embodiment step (D) is characterized in contacting said sample obtained in step (C) with magnetic affinity beads, thereby binding only the glycosylated heterologous polypeptide to said magnetic affinity beads and thereby separating said glycosylated heterologous polypeptide from other polypeptides from the cultivation by the removal of said sample and therewith not bound compounds in a single step. In one embodiment said glycosylated heterologous polypeptide accounts for more than 75% by weight of said bound polypeptide, or for more than 85% by weight of said bound polypeptide, or for more than 95% of weight of said bound polypeptide.

“Heterologous polypeptide” refers to a polypeptide, or a population of polypeptides, that do not exist naturally within a given host cell. DNA molecules heterologous to a particular host cell may contain DNA derived from the host cell species (i.e. endogenous DNA) so long as that host DNA is combined with non-host DNA (i.e. exogenous DNA). For example, a DNA molecule containing a non-host DNA segment encoding a polypeptide operably linked to a host DNA segment comprising a promoter is considered to be a heterologous DNA molecule. Conversely, a heterologous DNA molecule can comprise an endogenous structural gene operably linked with an exogenous promoter. A peptide or polypeptide encoded by a non-host DNA molecule is a “heterologous” peptide or polypeptide.

The sampling can either be done automatically or manually. In certain embodiments of the invention, the sampling step is performed automatically. The sample volume can range from 100 μl to 1000 μl. In one embodiment the sample obtained is purified. In one embodiment the method for the purification of the glycosylated heterologous polypeptide is selected from dialysis, fractionation on immunoaffinity or ion-exchange columns, ethanol precipitation, reverse phase high performance liquid chromatography (HPLC), chromatography on silica or on a cation-exchange resin, such as DEAE, chromatofocussing, sodium dodecylsulfate polyacrylamide gel electrophoresis (SDS-PAGE), ammonium sulfate precipitation, gel filtration, for example on SEPHADEX G-75®, or blotting on a protein binding membrane, like PVDF membranes, nylon membrane, or polytetrafluoroethylene (PTFE) membranes. A protease inhibitor such as phenyl methyl sulfonyl fluoride (PMSF) may be useful to inhibit proteolytic degradation during purification. One skilled in the art will appreciate that known purification methods which are also suitable for the glycosylated polypeptide of interest may require modification to account for changes in the character of the glycosylated polypeptide upon expression in a recombinant cell.

In one embodiment of the invention the purification method comprises binding of the recombinant glycosylated polypeptides to magnetic affinity beads and thereby allowing a rapid separation of the glycosylated polypeptides from impurities. With an iron core surrounded by agarose or inert polymer material, the beads behave like magnets when subjected to a magnetic field, yet retain no residual magnetism when the magnetic field is removed. The inventors of the current invention have found that this simplifies and shortens purification procedures, as no columns or centrifugation are required, e.g. in contrast to traditional agarose affinity methods (Smith, C., Nature Methods 2 (2005) 71-77). In particular, affinity binding and desorption kinetics take place in a fraction of the time required for slow column elution of solute-containing liquids, see e.g. Chaiken, I., et al., Analytical Biochemistry, 201 (1992) 197-210. Therefore, with the method according to the current invention a rapid determination of the momentary glycosylation profile of a glycosylated heterologous polypeptide produced in a cultivation can be performed, whereby the time required for said determination is extraordinarily short. Therefore, the current invention is providing in one aspect a method for the online or real-time determination of the glycosylation profile of a glycosylated heterologous polypeptide during its production in a cultivation allowing for the adjustment of the cultivation conditions during the cultivation, if required, in order to obtain the glycosylated heterologous polypeptide with a glycosylation profile according to the glycosylation profile of a reference sample. Another advantage of magnetic beads is that they can be used in a microtiter plate format, allowing the automation of the system described. Thus, another aspect of the current invention is an automated determination of the glycosylation profile of a glycosylated heterologous polypeptide during the cultivation process. The above mentioned advantages thus both increase the speed with which a glycosylation profile of a glycosylated polypeptide can be generated.

Antibodies can for example be purified by incubation with magnetic beads to which protein A, G, or L is bound. For this purpose, a truncated form of recombinant Protein A, G, or L is covalently coupled to a nonporous paramagnetic particle. Protein A exhibits high affinity for subclasses of IgG from many species including human, rabbit, and mouse. The protein is coupled through a linkage that is stable and leak resistant over a wide pH range. This permits the immunomagnetic purification of IgGs from ascites, serum, or cell culture supernatants. In one embodiment said IgGs are purified from cell culture supernatants. Glycosylated polypeptides in general can, for example, be purified by incubation with magnetic affinity beads to which deglycosylated antibodies specific for the glycosylated polypeptide, or lectins, or specific tags are bound. The use of deglycosylated antibodies as affinity molecules allows the later analysis of the glycosylation profile of the glycosylated polypeptide without the need to split up the antibody-glycosylated polypeptide complex first. Moreover, Protein A Magnetic Beads can be used to immunoprecipitate target proteins from crude cell lysates using selected deglycosylated primary antibody bound to said beads.

In further embodiments, step (D) includes a centrifugation step to remove cells and particulate cell debris from the culture broth. In still further embodiments, prior to step (E), step (D) includes removal of the solution surrounding the magnetic beads to which the glycosylated polypeptide is bound.

In step (E) of the method according to the invention, the glycans are released from the glycosylated polypeptide either enzymatically or chemically while the protein is still bound to the magnetic beads. The lack of an elution step, wherein the glycosylated polypeptide is released from the magnetic beads, markedly increases the speed with which the glycosylation profile can be determined in comparison to methods known in the art. It has been found that the elution of the glycosylated polypeptide from the magnetic beads prior to cleavage of the glycans, is not a necessary step for the method claimed and can be omitted without any disadvantage for the analysis of the glycosylation profile.

Embodiments for analyzing glycans of the glycosylated polypeptide basically include cleaving the glycans from the non-saccharide moiety using any chemical or enzymatic methods or combinations thereof that are known in the art. In certain embodiments of the invention, the chemical deglycosylation method is hydrazinolysis. In other embodiments, the glycans can be removed from the glycosylated polypeptides by alkali borohydride treatment or trifluoro methanesulfonic acid (TFMS) treatment. In the latter case the deglycosylated protein can be redissolved in 8 M urea prior to further analysis.

Enzymatic methods for the glycan cleavage include methods that are specific to N- or O-linked sugars. These enzymatic methods include the use of Endoglycosidase, exemplarily selected from Endoglycosidase F (EndoF), or Endoglycosidase H (Endo H), or Endoglycosidase N (Endo N), or Endoglycosidase D (Endo D), or N-Glycanase F (PNGaseF), or combinations thereof. N-Glycosidase F, also known as PNGase F, is an amidase that cleaves between the innermost GlcNAc and asparagine residues of high mannose, hybrid, and complex oligosaccharides from N-linked glycosylated polypeptides. In certain embodiments of the invention, PNGaseF, which cleaves all mammalian N-glycan structures, is used for release of N-glycans.

The glycans analyzed by the method according to the invention can also be in an additional step contacted with a glycan-degrading enzyme. In one embodiment step (E) comprises in addition contacting said released glycans with a glycan-degrading enzyme. Examples of glycan-degrading enzymes are known in the art and include exoglycosidases, or N-glycanase, or neuraminidase I, or neuraminidase III, or galactosidase I, or N-acetyl-glucosaminidase I, or alpha-fucosidase II and III, or sialidase, or mannosidase, or a combination thereof.

In further embodiments, this step (E) further includes contacting the glycans with more than one glycan-degrading enzyme either sequentially or simultaneously. In some embodiments, the enzymatic digestion is sequential, such that not all (mono-) saccharides are removed immediately. The digested glycans can be analyzed after each digestion step to obtain a glycosylation profile (see for example WO 2006/114663).

In still further embodiments trypsin, or Endoproteinase, like Arg C, Lys C and Glu C, for example, can be used to obtain a peptide digest prior to determination of the glycosylation pattern of the glycosylated polypeptide of interest.

In another embodiment, the deglycosylation step includes denaturing and/or unfolding of the glycosylated heterologous polypeptide prior to cleavage of the glycan. In another embodiment, the denaturing agent is selected from a detergent, or urea, or guanidinium hydrochloride, or heat. In a further embodiment, the glycosylated heterologous polypeptide is reduced following the denaturation. In yet another embodiment, the glycosylated heterologous polypeptide is reduced with a reducing agent. The reducing agent in certain embodiments is selected from DTT or β-mercaptoethanol, or TCEP. In a further embodiment, the glycosylated heterologous polypeptide is alkylated with an alkylating agent following the reduction. The alkylating agent in certain embodiments is selected from iodoacetic acid or iodoacetamide. Iodination and/or reduction of the proteins can be performed with the proteins still bound to the magnetic beads.

In step (F) of the method for the production of a recombinant protein, the enzymatically or chemically released glycans are purified for further analysis. In certain embodiments of the invention, everything but the glycans is removed from the sample. In certain embodiments of the invention, purification of the glycans is performed by reverse phase liquid chromatography or cation exchange chromatography. Samples are for example purified with commercially available resins or chromatographic materials and/or cartridge systems used to separate glycans and proteins for clean-up after chemical cleavage or enzymatic digestion. Such resins, materials and cartridges include ion exchange resins and purification columns, such as GlycoClean H, S, and R cartridges. In some embodiments GlycoClean S in combination with GlycoClean H is used for purification. This solid-phase extraction (SPE) cartridge contains a porous graphitic carbon (PGC) matrix useful for removal of proteins and desalting of the released glycans prior to the mass spectrometry (MALDI-TOF) analysis. In other embodiments a strong cation-exchange resins (AG® 50W-X2) is used.

By employment of different purification methods different materials may be suited. Ion exchange resins for example are available under different names and from a multitude of companies such as cation exchange resins Bio-Rex® (e.g. type 70), Chelex® (e.g. type 100), Macro-Prep® (e.g. type CM, High S, 25 S), AG® (e.g. type 50W, MP) all available from BioRad Laboratories, WCX 2 available from Ciphergen, Dowex® MAC-3 available from Dow chemical company, Mustang C and Mustang S available from Pall Corporation, Cellulose CM (e.g. type 23, 52), hyper-D, partisphere available from Whatman plc., Amberlite® IRC (e.g. type 76, 747, 748), Amberlite® GT 73, Toyopearl® (e.g. type SP, CM, 650M) all available from Tosoh Bioscience GmbH, CM 1500 and CM 3000 available from BioChrom Labs, SP-Sepharose™, CM-Sepharose™ available from GE Healthcare, Poros resins available from PerSeptive Biosystems, Asahipak ES (e.g. type 502C), CXpak P, IEC CM (e.g. type 825, 2825, 5025, LG), IEC SP (e.g. type 420N, 825), IEC QA (e.g. type LG, 825) available from Shoko America Inc., 50W cation exchange resin available from Eichrom Technologies Inc., and such as e.g. anion exchange resins like Dowex® 1 available from Dow chemical company, AG® (e.g. type 1, 2, 4), Bio-Rex® 5, DEAE Bio-Gel 1, Macro-Prep® DEAE all available from BioRad Laboratories, anion exchange resin type 1 available from Eichrom Technologies Inc., Source Q, ANX Sepharose 4, DEAE Sepharose (e.g. type CL-6B, FF), Q Sepharose, Capto Q, Capto S all available from GE Healthcare, AX-300 available from PerkinElmer, Asahipak ES-502C, AXpak WA (e.g. type 624, G), IEC DEAE all available from Shoko America Inc., Amberlite® IRA-96, Toyopearl® DEAE, TSKgel DEAE all available from Tosoh Bioscience GmbH, Mustang Q available from Pall Corporation. In a membrane ion exchange material the binding sites can be found at the flow-through pore walls and not hidden within diffusion pores allowing the mass transfer via convection than diffusion. Membrane ion exchange materials are available under different names from some companies such as e.g. Sartorius (cation: Sartobind™ CM, Sartobind™ S, anion: Sartobind™ Q), or Pall Corporation (cation: Mustang™ S, Mustang™ C, anion: Mustang™ Q), or Pall BioPharmaceuticals. Preferably the membrane cation exchange material is Sartobind™ CM, or Sartobind™ S, or Mustang™ S, or Mustang™ C.

In still other embodiments, the glycans are purified by dialysis or by precipitating concomitant proteins with ethanol or acetone and removing the supernatant containing the glycans. Other experimental methods for removing the proteins, detergent (from a denaturing step), or/and salts include methods known in the art.

In still other embodiments, the glycans are purified by affinity binding of the glycans to magnetic beads or binding to magnetic reverse phase beads (like C18-beads), removal of salts and proteins, and subsequent elution of the glycans from the beads.

General chromatographic methods and their use which are also applicable in this invention are known to a person skilled in the art. See for example, Chromatography, 5^{th} edition, Part A: Fundamentals and Techniques, Heftmann, E. (ed.), Elsevier Science Publishing Company, New York, (1992); Advanced Chromatographic and Electromigration Methods in Biosciences, Deyl, Z. (ed.), Elsevier Science BV, Amsterdam, The Netherlands, (1998); Chromatography Today, Poole, C. F., and Poole, S. K., Elsevier Science Publishing Company, New York, (1991); Scopes, Protein Purification: Principles and Practice (1982); Sambrook, J., et al. (ed.), Molecular Cloning: A Laboratory Manual, Second Edition, Cold Spring Harbor Laboratory Press, Cold Spring Harbor, N.Y., 1989; or Current Protocols in Molecular Biology, Ausubel, F. M., et al. (eds), John Wiley & Sons, Inc., New York.

For the purification of recombinantly produced heterologous immunoglobulins e.g. often a combination of different column chromatographical steps is employed. Generally a Protein A affinity chromatography is followed by one or two additional separation steps. The final purification step is a so called “polishing step” for the removal of trace impurities and contaminants like aggregated immunoglobulins, residual HCP (host cell protein), DNA (host cell nucleic acid), viruses, or endotoxins. For this polishing step often an anion exchange material in a flow-through mode is used.

The affinity material may e.g. be a protein A affinity material, a protein G affinity material, a hydrophobic charge induction chromatography material (HCIC), or a hydrophobic interaction chromatography material (HIC, e.g. with phenyl-sepharose, aza-arenophilic resins, or m-aminophenylboronic acid). Preferably the affinity material is a Protein A material or a HCIC material.

In step (G) of the method, the glycosylation profile of the recombinantly expressed protein is determined. Several techniques are available for the determination of a glycosylation profile of a glycosylated heterologous polypeptide (glycoprotein) and any analytic method for analyzing the glycosylation pattern of a glycosylated polypeptide can be employed. The term “analyzing the glycosylation pattern” means to obtain data that can be used to determine the glycosylation sites, or/and the glycosylation site occupancy, or/and the identity, or/and the structure, or/and the composition, or/and the quantity of the glycan or/and non-saccharide moiety of the glycoprotein as well as the identity and quantity of the specific glycoform.

Methods which can be used for analysis of the glycosylation pattern can be selected from mass spectrometry, nuclear magnetic resonance (NMR, such as 2D-NMR), chromatographic methods, or electrophoretical methods. Examples of mass spectrometric methods are FAB-MS, LC-MS, LC-MS/-MS, MALDI-MS, MALDI-TOF, TANDEM-MS, FTMS, or electrospray-ionization-quadrupole-time-of-flight-MS (ESI-QTOF-MS; see e.g. Müthing, J., et al., Biotech. Bioeng. 83 (2003) 321-334). NMR methods are, for example, COSY, TOCSY, or NOESY. Electrophoretical methods are, for example, CE-LIF (see e.g. Mechref, Y., et al., Electrophoresis 26 (2005) 2034-2046). In certain embodiments of the invention, the chromatographic method is high performance anion exchange chromatography with pulsed amperometric detection (HPAEC; see for example Field, M., et al., Anal. Biochem. 239 (1996) 92-98), weak ion exchange chromatography (WAX), gel permeation chromatography (GPC), high performance liquid chromatography (HPLC), normal phase high performance liquid chromatography (NP-HPLC), reverse phase HPLC (RP-HPLC), or porous graphite carbon HPLC (PGC-HPLC).

In other embodiments the glycans are quantified by using calibration curves of glycan standards of known structure, and/or composition, and/or identity.

Other methods that can be used to analyze the saccharide composition of the glycans once released from the protein include procedures involving the labeling of the saccharides with chemical or fluorescent tags. Such methods are fluorescence assisted carbohydrate electrophoresis (FACE), HPLC, or capillary electrophoresis (CE, see e.g. Rhomberg, E., et al., Proc. Natl. Acad. Sci. USA 95 (1998) 4176-4181).

In some embodiments, the measuring of the glycosylation profile with HPLC can be complemented with a mass spectrometry measurement. Complementary mass spectrometry data, such as MALDI, ESI, or LC/MS can serve, for example, for validation of HPLC measured glycosylation profiles as a separate orthogonal technique able to resolve the structures of more complex glycans when a sufficient amount of sample is available.

In certain embodiments of the invention, the analytic method for the characterization of the glycans includes the use of MALDI-TOF MS. Therein the relative intensities of the unmodified glycan signals represent their relative molar proportions in the sample, allowing relative quantification of both neutral and sialylated glycan signals. MALDI MS techniques for the analysis of oligosaccharides have also been described (Juhasz, P., and Biemann, K., Carbohydr. Res. 270 (1995) 131-147; Venkataraman, G., et al., Science 286 (1999) 537-542; Rhomberg, E., et al., Proc. Natl. Acad. Sci. USA 95 (1998) 4176-4781; Harvey, D. J., Mass. Spectrom. Rev. 18 (2000) 349-450).

Experimental conditions according to the present invention are described in the Examples listed below.

The matrix compounds and procedures of sample preparation have significant influence on the ion response of analytes in MALDI MS. In certain embodiments of the invention, the matrix preparation is 2,5-dihydroxy benzoic acid (DHB). In some embodiments the matrix preparation is caffeic acid with or without spermine. In other embodiments, the matrix preparation is DHB with spermine. The spermine, for example, can be in the matrix preparation at a concentration of 300 mM. The matrix preparation can also be a combination of DHB, spermine, and acetonitrile. MALDI MS can also be performed in the presence of Nafion and ATT (6-aza-2-thiothymin). In still further embodiments, the following matrixes can be used: α-cyano-4-hydroxy-cinnamic acid (4-HCCA), 4-hydroxy-3-methoxycinnamic acid (FA), 3-hydroxypicolinic acid (HPA), 5-methoxysalicyclic acid (MSA), DHB/MSA, DHB/MSA/Fucose, DHB/Isocarbostyril (HIC), or those described in U.S. Pat. No. 5,045,694 and U.S. Pat. No. 6,228,654. In addition to matrices, the sample preparation procedures, such as concentration of sodium chloride (for not derivatized oligosaccharides), evaporation environment (in air or vacuum), and re-crystallization conditions (using different organic solvents) can affect sensitivity of the overall analysis and thus have to be controlled.

Additionally, when using MALDI-TOF MS to analyze the samples, instrument parameters can also be modified. These parameters include guide wire voltage, accelerating voltage, grid values, or/and negative versus positive mode. In certain embodiments of the invention, for MALDI-TOF MS of unmodified glycans in positive ion mode, optimal mass spectrometric data recording range according to the present invention is over m/z 200 and for improved data quality over m/z 1000. For MALDI-TOF mass spectrometry of unmodified glycans in negative ion mode, optimal mass spectrometric data recording range according to the present invention is over m/z 200, and over m/z 1000 for improved data quality.

The preferred ranges depend on the sizes of the sample glycans. Samples with high branching or polysaccharide content or high sialylation levels are preferably analyzed in ranges containing higher upper limits as described for negative ion mode. The limits are preferably combined to form ranges of maximum and minimum sizes or lowest lower limit with lowest higher limit, and the other limits analogously in order of increasing size.

The glycan analysis of the mass spectrometry spectra includes determining the glycosylation site occupancy, the identity, the structure, the composition and/or the quantity of the glycan and/or non-saccharide moiety of the glycosylated polypeptide as well as the identity and quantity of a specific glycoform. For this purpose glycan libraries are used. In some embodiments, a combined analytical-computational platform is used to achieve a thorough characterization of glycans.

In another embodiment, the method further includes recording the pattern in a computer-generated data structure.

In step (H) of the method, the glycosylation profile of the glycosylated polypeptide is compared with a desired pre-determined reference glycosylation profile. This can either be done manually or automatically. In certain embodiments of the invention, an automatic analysis by an Excel macro is used.

In step (I) of the method, the cell clone of step (A) is cultivated under modified cultivation conditions in accordance to the results obtained in step (G), i.e. when the glycosylation profile determined in step (G) differs from the pre-determined reference glycosylation profile. Then, steps (C) to (H) are repeated several times, in one embodiment 2 to 20 times, in another embodiment 2 to 10 times, or daily, in order to finally obtain a glycosylated polypeptide in accordance with the pre-determined reference glycosylation profile. For example, if it is detected that the glycosylated polypeptide contains only low amounts of a certain monosaccharide, then specifically this monosaccharide is added to the culture medium (see e.g. U.S. Pat. No. 6,673,575).

The modification of culture conditions in step (I) of the method according to the invention are selected from alterations of types and concentrations of provided nutrient(s), buffer, additives, carbohydrates, or ammonium, or concentration of the dissolved oxygen, or osmolality, or pH value, or temperature, or cell density, or growth stage. All these parameter can be altered either alone or in combination in order to obtain a glycosylated heterologous polypeptide with a glycosylation pattern of the reference glycosylated polypeptide. All of these parameters can be controlled either manually or automatically. The osmolality e.g. is modified by changing the concentration of sodium chloride, different amino acids, hydrolyzates or sodium hydroxide, the pH value is modified by the addition of acid or base, e.g. to be of from pH 6.9 to pH 7.2, and the ammonium concentration is regulated by glutamine and/or NH₄Cl addition for example.

Purification of the glycosylated polypeptide, deglycosylation and purification of the glycans, as well as subsequent MALDI-TOF MS analysis can be performed in one embodiment in a high-throughput manner in microtiter plates, enabling the automation of the system described. The high-throughput format can use standard multiwell formats such as 48 well plates or 96 well plates. For example the method according to the invention may be used in a high throughput format using a multiwell micro plates and a micro plate reader (e.g. a Tecan Safire™, Infinite™, or Sunrise™, Tecan Trading AG, CH) to follow multiple cultivations in parallel.

Surprisingly, it is possible by using the method according to the present invention to decrease the time required for the determination of the glycosylation profile of a glycosylated heterologous polypeptide in comparison to procedures known in the art. In particular, the release of glycans from the glycosylated polypeptides still bound to magnetic affinity beads efficiently decreases the analysis time. The method according to the present inventions enables the adjustment of the culture conditions during fermentation to obtain the desired glycosylation profile. Further, the method of the present invention can be performed in a 96 well microtiter plate format such that it can be fully automated, for example by means of a Tecan robotic system.

The highly dynamic process of posttranslational glycosylation of proteins, in which rapid changes in the carbohydrate structures occur in response to cellular signals or cellular stages, result in key informational markers of some serious human diseases. For example, it is known that carbohydrate structures in patients with rheumatoid arthritis can be strongly altered and that specific carbohydrates are used as tumor-associated markers in pancreatic and colon cancers (Nishimura, S. I., et al., Angew. Chem. Int. Ed 44 (2005) 91-96).

The present invention, thus, also relates to a method suitable for use in diagnosis comprising determining and/or quantifying a glycosylation marker of a disease. Said method comprises steps of the method for the production of a glycosylated polypeptide claimed.

Therefore, an aspect of the current invention is a method for determining and/or quantifying a glycosylation marker comprising the steps of:
(A) contacting a sample obtained from a patient containing a glycosylated polypeptide with magnetic affinity beads, thereby binding said glycosylated polypeptide to said magnetic affinity beads,
(B) removing the magnetic affinity beads with the bound glycosylated polypeptide from the sample,
(C) releasing the glycans from the glycosylated polypeptide bound to the magnetic affinity beads, without the polypeptide being released from the magnetic affinity beads,
(E) determining the amount of the glycosylation marker, and
(F) comparing the determined amount of the glycosylation marker with a reference amount of the glycosylation marker.

In another embodiment comprises the method prior to step (A) the step (A-1) of purifying the sample by applying it to one or more chromatography columns. In one embodiment the method comprises a step (D) after step (C) and prior to step (E) of (D) purifying the released glycans.

The sample to be analyzed with the above method can, for example, be a sample of a body tissue or of a body fluid such as whole serum, blood plasma, synovial fluid, urine, seminal fluid or saliva, sputum, tears, CSF, feces, tissues or cells. The glycosylated polypeptides to be analyzed can be the total glycosylated polypeptides in the sample, a fraction or only a single glycosylated polypeptide, known as diagnostic marker(s) for specific disease(s).

The term “glycosylation marker” as used within this application denotes a polysaccharide composed of at least three monosaccharides whose amount is altered, either enhanced or decreased, in certain diseases.

In one embodiment, the pattern associated with a diseased state is a pattern associated with cancer, such as prostate cancer, melanoma, bladder cancer, breast cancer, lymphoma, ovarian cancer, lung cancer, colorectal cancer or head and neck cancer. In other embodiments, the pattern associated with a diseased state is a pattern associated with an immunological disorder, a neurodegenerative disease, such as a transmissible spongiform encephalopathy, Alzheimer's disease or neuropathy, inflammation, rheumatoid arthritis, cystic fibrosis, or an infection (viral or bacterial infection). In an other embodiment the method is a method for monitoring prognosis and the known pattern is associated with the prognosis of a disease. In yet another embodiment, the method is a method of monitoring drug treatment and the known pattern is associated with the drug treatment.

The measured glycosylation profile can in one embodiment be compared with a control glycoprofile of a second subject supposed to be healthy to determine one or more glycosylation markers of a specific disease. Comparing the glycosylation profiles can involve in one embodiment comparing peak ratios in the profiles. When more than one glycosylation marker is identified, one can select one or more of the markers that have the highest correlation with one or more parameters of the subject diagnosed with a specific disease (see also US 2006/0270048).

Different methods are well established and widespread used for protein recovery and purification, such as affinity chromatography with microbial proteins (e.g. protein A or protein G affinity chromatography), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M. A., Appl. Biochem. Biotech. 75 (1998) 93-102).

The following examples and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Exemplary scheme of the method according to the invention for the recombinant production of an antibody A with a defined glycosylation profile.

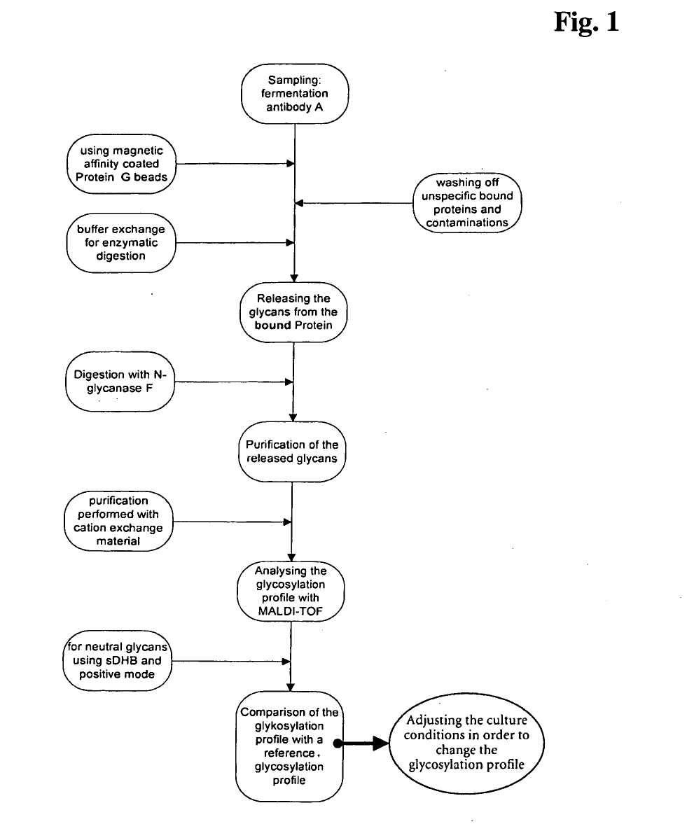

FIG. 2 MALDI-TOF MS of the PNGase F-released oligosaccharides from a sample during the production of a monoclonal anti-CCR5 antibody. The N-linked oligosaccharides of the antibody were released and analyzed by MALDI-TOF MS in the positive ion mode using a DHB matrix as described in Example 2.

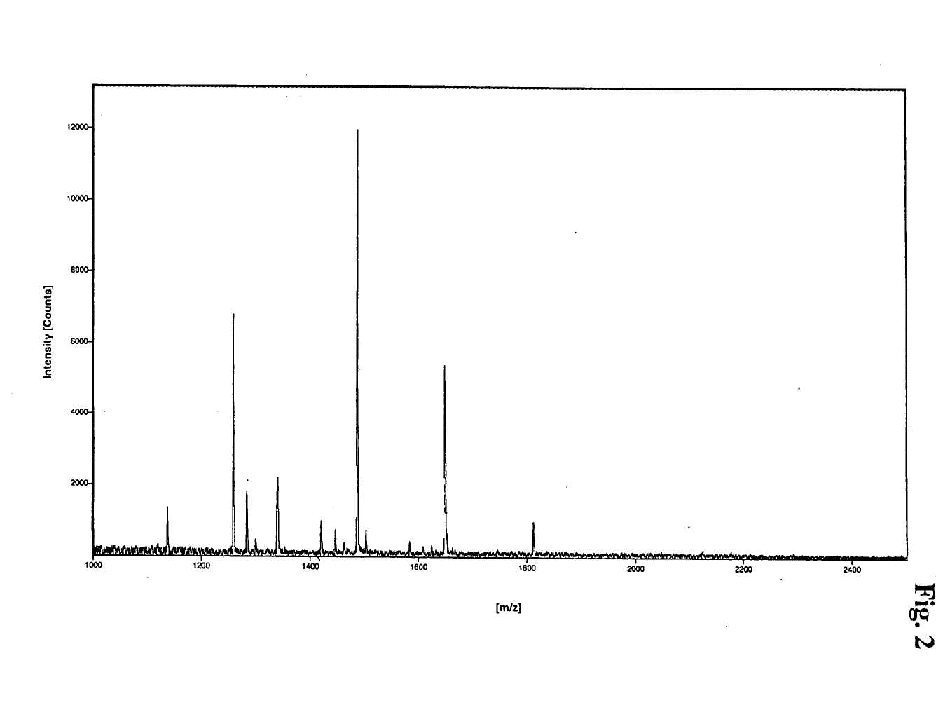

FIG. 3 Follow up of selected glycans during the production of a monoclonal anti-CCR5 antibody in fed-batch culture without a change of the cultivation conditions during the cultivation. At different time points the glycosylation profile of the produced antibody bound to magnetic affinity beads was determined by MALDI-TOF MS after PNGase F digestion. The relative amount of selected different glycan structures during fermentation is shown. ▪ Man5, Man6, ▴ Man7, and Man8.

FIG. 4 Follow up of selected glycans of during the production of a monoclonal anti-CCR5 antibody in fed-batch culture with a change of the cultivation pH during the cultivation. At different time points the glycosylation profile of the produced antibody bound to magnetic affinity beads was determined by MALDI-TOF MS after PNGase F digestion. During the cultivation the pH was changed from 7.2 to 6.9 at day 8. The relative amount of selected different glycan structures during fermentation is shown. ▪ Man5, Man6, ▴ Man7 and Man8.

### EXAMPLES

### Example 1

Production of Monoclonal Anti-CCR5 Antibody

Cells producing a recombinant anti-CCR5 antibody were generated according to established procedures (see e.g. Olson, W. C., et al., J. Virol. 73 (1999) 4145-4155; Samson, M., et al., J. Biol. Chem. 272 (1997) 24934-24941; EP 1322332; WO 2006/103100; WO 2002/083172) and cultured in serum-free medium (fed-batch culture) in a controlled bioreactor environment (see for example Meissner, P. et al., Biotechnol. Bioeng. 75 (2001) 197-203). The temperature was maintained at 37° C., pH was set to 6.9 or 7.2, and dissolved oxygen concentration was maintained at 35%. At the beginning of the fermentation the cell density was 5×10⁵ cells/ml. At specific time points during the fermentation, samples containing the recombinant antibodies were removed from the culture for analysis.

### Example 2

Analysis of the Glycosylation Profile of Antibody Containing Samples

For each sample, 300 μl of magnetic affinity protein G coated beads (MagnaBind Protein G, Pierce) were washed three times with 250 μl of Protein G IgG Binding buffer (Protein G IgG Binding buffer, Pierce). After each washing step, the binding buffer was completely removed. Then, 200 μl of each sample and 100 μl of Binding buffer were added to the prepared magnetic affinity beads. The solutions were then incubated for one hour at room temperature. Afterwards, the liquid was completely removed. The incubated beads were then washed twice with 250 μl of a solution containing 2 mM TRIS-HCl and 150 mM NaCl at pH 7.0 to remove unspecific bound material. Afterwards, the beads were washed three times with ultra pure water. After each washing step, the liquid was completely removed. Then, 60 μl of ultra pure water and 2 μl of PNGase F solution (100 mU dissolved into 100 μl of ultra pure water) were added to the beads. The digestion was performed at 37° C. for four hours. After the digestion, 2.2 μl of a 1.5 M acetic acid solution were added to 20 μl of the sample and incubated for a further three hours at room temperature to convert glycosylamine into the reduced form. The glycans were then purified by use of a weak cation exchange material. For each sample a separate column was prepared. The cation exchange material (AG® 50W-X8 Resin, BIO-RAD) was washed three times with ultra pure water. 900 μl of the washed resin were then filled into a chromatography spin column (Micro Bio-Spin, BIO-RAD). The columns were centrifuged for 1 min at 1,000×g to remove excess water. Then, 22.2 μl of each sample was loaded onto the surface of the prepared columns. The column was again centrifuged for 1 min at 1,000×g. The liquid now contained the purified glycostructures. The samples were then mixed with sDHB matrix (1.6 mg of 2,5-dihdroxybenzoic acid and 0.08 mg of 5-methoxysalicylic acid were dissolved in 125 μl of ultra pure ethanol and 125 μl of 10 mM NaCl solution) at a ratio of 1:2. 1.5 μl of the mix was then directly spotted onto the MALDI-TOF target. The samples were allowed to dry for the subsequent MALDI-TOF analysis. A MALDI-TOF mass spectrometer in the positive reflector mode was used for the measurements.

Results:

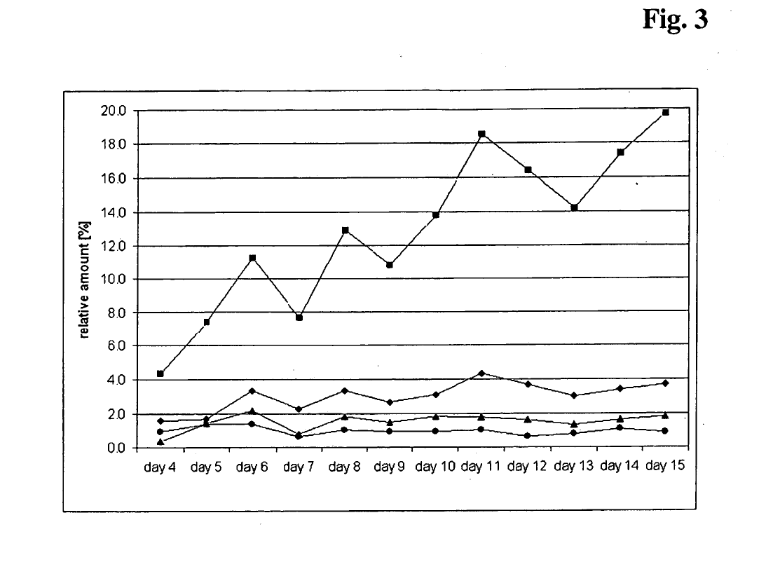

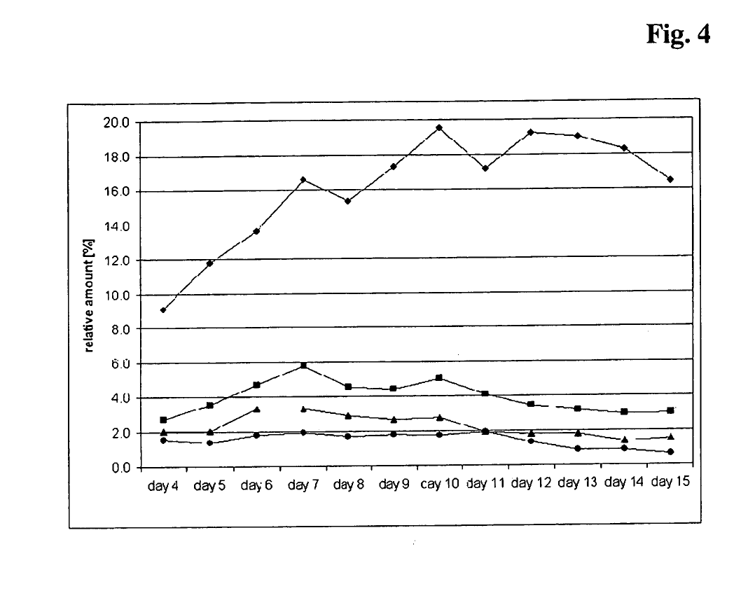

In FIG. 3 the course of selected glycans during the production of a monoclonal anti-CCR5 antibody in a fed batch culture is shown. The pH was set to 6.9. The content of Man5 increased steadily in the course of fermentation resulting in a relative amount of about 20% after fifteen days of cultivation. In FIG. 4, the glycosylation profile of the same antibody is shown under altered environmental conditions: The pH was set to 7.2 at the beginning of the fermentation. Eight days after start of the fermentation, the pH was changed to 6.9. The relative amount of Man5 decreased during the last days of fermentation, resulting in a lower final relative amount of Man5 (16%) compared to the data obtained in the experiment shown in FIG. 3.

1. (canceled)

2. A method for the recombinant production of a glycosylated heterologous polypeptide comprising the steps of:
(A) providing a cell comprising a nucleic acid encoding said heterologous polypeptide,
(B) cultivating said cell under conditions suitable for the expression of said heterologous polypeptide,
(C) obtaining a sample from the cultivation medium of said cell,
(D) contacting said sample with magnetic affinity beads under conditions suitable for the binding of the heterologous polypeptide to the magnetic affinity beads,
(E) releasing the glycans from said heterologous polypeptide bound to said magnetic affinity beads without releasing said heterologous polypeptide,
(F) purifying said released glycans of step (E),
(G) determining the glycosylation profile of the heterologous polypeptide,
(H) comparing the determined glycosylation profile with a reference glycosylation profile,
(I) adjusting the culture conditions in accordance with the result obtained in step (H), and optionally continuing with the cultivation and step (J), or stopping the cultivation and obtaining said glycosylated heterologous polypeptide, and
(J) repeating steps (C) to (H) to obtain the glycosylated heterologous polypeptide.

3. The method according to claim 2, characterized in that said glycosylated heterologous polypeptide is an immunoglobulin.

4. The method according to claim 3, characterized in that said magnetic affinity beads are magnetic affinity beads with protein A, G, or L bound thereto.

5. The method according to claim 2, characterized in that said releasing the glycans is an enzymatically releasing by an N-glycosidase.

6. The method according to claim 2, characterized in that said releasing the glycans is a chemically releasing by hydrazinolysis.

7. (canceled)

8. The method according to claim 2, characterized in that said determining of the glycosylation profile of the heterologous polypeptide is by matrix-assisted laser desorption ionization time-of-flight mass spectrometry analysis or quantitative high performance liquid chromatography separation of the released and purified glycans.

9. The method according to claim 2, characterized in that steps (D) to (G) are performed in a high-throughput format using microtiter plates.

10. The method according to claim 2, characterized in that said adjusting of the culture conditions comprises one or more alterations in:
(i) the concentration of nutrients, carbohydrates, additives, buffer, ammonium, or dissolved oxygen,
(ii) the osmolality, pH value, temperature, or cell density, and/or
(iii) the growth state.

11. The method according to claim 2, characterized in that it comprises an additional step (K):
(K) recovering the glycosylated heterologous polypeptide from the culture medium or the cells.

12. The method according to claim 11, characterized in that it comprises after step (K) an additional step (L):
(L) purifying said heterologous polypeptide.

13. The method according to claim 2, characterized in that said step (E) is:
(E) releasing the glycans from the heterologous polypeptide and recovery of the released glycans without the release of the heterologous polypeptide from the magnetic affinity beads by removing the magnetic affinity beads with the bound heterologous immunoglobulin from the sample.

14. The method according to claim 2, characterized in that said step (F) is:
(F) purifying the glycans released in (E) by a high performance liquid chromatography on a cation exchange resin or on a reversed phase.

15. The method according to claim 2 characterized in that said cell is a CHO cell, or a BHK cell, or a HEK cell.

16. (canceled)

17. (canceled)

18. (canceled)

19. The method according to claim 2, characterized in that the concentration of the cells after the growth phase is more than 1×10⁶ cells/ml, or more than 5×10⁶ cells/ml, or the cells have a dry cell weight of more than 100 g/l, or more than 200 g/l.

20. The method according to claim 2, characterized in that the total concentration of all sugars during the cultivation is of from 0.1 g/l to 10 g/l.

21. The method according to claim 20, characterized in that the total concentration of all sugars is of from 2 g/l to 6 g/l in the culture medium.

22. The method according to claim 2, characterized in that said glycosylated heterologous polypeptide accounts for more than 75% by weight of said bound polypeptide in step (B) or (D), respectively.

23. The method according to claim 2, characterized in that step (E) comprises in addition contacting said released glycans with a glycan-degrading enzyme.

24. The method according to claim 2, characterized in that said deglycosylation step (E) comprises denaturing and/or unfolding of the glycosylated heterologous polypeptide prior to cleavage of the glycan.

25. The method according to claim 24, characterized in that said glycosylated heterologous polypeptide is reduced following the denaturation.

26. (canceled)
